SEQUENCE LISTING (6 sequences)

SEQ ID NO: 1 - DNA, 44 bp, Campylbacter jejuni
atgagtgaatgccccttttttccaaaaccttataaaaataaagc

SEQ ID NO: 2 - DNA, 45 bp, Campylbacter jejuni
tcagtggtggtggtggtggtgtagctttcttttgctaaattttat

SEQ ID NO: 3 - protein, 453 aa, Campylbacter jejuni
MSECPFFPKPYKNKASTLLTFLLKRRSWLDGLYERSYKMQTGYVKMPNFDLYVINDTKEVKRMMVDEVREFPKSAFLHELLSPLLGESIFTTNGEVWKKQRELLRPSFEMTRINKVFNLMSEAVADMMDRFSKYPNHAVIEVDEAMTFITADVIFRTIMSSKLDEEKGKKILNAFVTFQEQSVHTAMRRMFRFPKWLSYVLGDCKRAKAGDVIRQVLSDIIKPRYDMADNAEFEDILGSLLLVVDADTNKRFSFEEILDQVAMLFLAGHETTASSLTWTLYLLSLYPKEQEKAYEEITQVLQGGVIEISHLRQFKYLTNIFKESLRLYPPVGFFAREAKKDTQVRDKLIKKGSGVVIAPWLIHRHEEFWTNPHGFNPSRFEGEYKKDAYLPFGVGERIFIGQGFAMQEAILILANILKTYKLELEEGFVPDVVGRLTVRSANGMRIKFSKRKL

SEQ ID NO: 4 - protein, 10 aa, Campylbacter jejuni
FGVGERIFIG

SEQ ID NO: 5 - protein, 453 aa, Campylbacter jejuni
MSECPFFPKPYKNKASTLLTFLLKRRSWLDGLYERSYKMQTGYVKMPNFDLYVINDTKEVKRMMVDEVREFPKSAFLHELLSPLLGESIFTTNGEVWKKQRELLRPSFEMTRINKVFNLMSEAVADMMDRFSKYPNHAVIEVDEAMTFITADVIFRTIMSSKLDEEKGKKILNAFVTFQEQSVHTAMRRMFRFPKWLSYVLGDCKRAKAGDVIRQVLSDIIKPRYDMADNAEFEDILGSLLLVVDADTNKRFSFEEILDQVAMLFLAGHETTASSLTWTLYLLSLYPKEQEKAYEEITQVLQGGVIEISHLRQFKYLTNIFKESLRLYPPVGFFAREAKKDTQVRDKLIKKGSGVVIAPWLIHRHEEFWTNPHGFNPSRFEGEYKKDAYLPFGVGERICIGQGFAMQEAILILANILKTYKLELEEGFVPDVVGRLTVRSANGMRIKFSKRKL

SEQ ID NO: 6 - protein, 10 aa, Campylbacter jejuni
FGVGERICIG

(19) United States
(12) Patent Application Publication
(10) Pub. No.: US 2025/0360193 A1
(43) Pub. Date: Nov. 27, 2025

(54) VACCINE AGAINST CAMPYLOBACTER JEJUNI

(71) Applicant: Envirotech Innovative Products Limited, Dublin (IE)

(72) Inventor: Patrick WARD, Dublin (IE)

(21) Appl. No.: 18/836,121

(22) PCT Filed: Feb. 10, 2022
(86) PCT No.: PCT/EP2022/053293

§ 371 Date: Aug. 6, 2024

(51) Int. Cl. A61K 39/02; A61K 39/00; A61P 37/04; C07K 14/205; C07K 16/12

(52) U.S. Cl. CPC A61K 39/105; A61P 37/04; C07K 14/205; C07K 16/121; A61K 2039/542; A61K 2039/545; A61K 2039/552

(57) ABSTRACT

The invention provides a polypeptide that is antigenic in a host. The invention also provides a vaccine for use in reducing or preventing Campylobacter colonization in a host. The vaccine comprises a polypeptide of the present invention and/or antibodies against the polypeptide of the present invention. The host may be a human, cow, sheep, goat, and chicken. The invention has particular application for reducing or preventing Campylobacter jejuni colonization in poultry. The invention also provides a vaccine for use in reducing or preventing campylobacteriosis; and a vaccine composition comprising the polypeptide of the present invention; or antibodies raised against the polypeptide of the present invention, in association with a pharmaceutically acceptable vehicle useful for inducing an immune response in a host.

### INTRODUCTION

Chicken meat is a major source of campylobacters, which grow profusely in the gut of broiler chickens (a broiler chicken (Gallus gallus domesticus) is bred and raised for meat production), leading to over 85% of UK carcasses being contaminated. The European Food Safety Authority (EFSA) has stated that a 1,000-fold reduction of the numbers of Campylobacter in broiler intestines, at slaughter, would reduce the public health risk by at least 90%, since Campylobacter contamination of broiler meat by the gut flora would then fall below the infectious dose for human beings. The development of an effective Campylobacter vaccine for broilers would see this target for risk reduction met as the meat from vaccinated birds would fall below the infectious dose for human beings. Recently significant progress has been made in identifying bacterial proteins that will promote colonization of broilers (Sima, et al., 2018; Song, Malmuthuge, Steele, & Guan, 2018).

Herein, we have developed a new vaccine using proteins or protein fragments of proteins involved in bacterial respiration (Kelly, 2001). Herein, we have identified novel proteins involved in vital metabolic processes which, once attacked by antibodies produced by the immune system, will result in bacterial death or in reduced pathogen abilities to maintain and colonise the avian gut. This will allow the production of specific vaccine targets without contamination by similar products that might be synthesized in the original organism. Another advantage of this technology is that only biologically active proteins are produced allowing a tighter control of the effective dosage required.

Campylobacter is not known to cause diarrhoeal symptoms in poultry and, in order to test for disease prevention, the rabbit intestinal loop model will be used herein (Newell, 2001). This animal model will allow us to not only show the effect on intestinal colonization but also to investigate its ability to prevent mucosal inflammation. The evidence of the importance in pathogenesis will come from the evaluation of the rabbit ileal loops infected with Campylobacter. This pathogen will multiply in the intestinal epithelium and produce an inflammatory reaction of the intestinal mucosa in non-vaccinated rabbits. This animal system resembles the natural disease of human and will be useful as a rapid and inexpensive model to study the process of infectivity, to assess enteropathogenicity, to elucidate the effectiveness of pharmacological agents, and to test the possible efficacy of vaccine products.

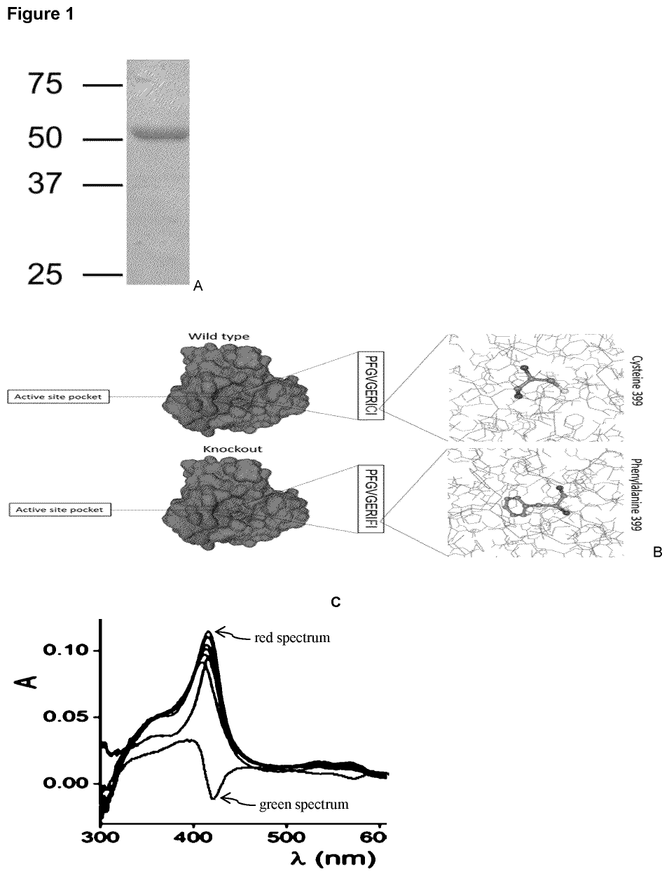

Neal-Mckinney et al., 2014 suggested that the vaccination of chickens with certain C. jejuni surface exposed colonisation proteins would reduce the ability of C. jejuni to colonise chickens. They tested this in an artificial infection model, by vaccinating chicken through injecting them with recombinant C. jejuni peptides from CadF, FlaA, FlpA, CmeC and a CadF-FlaA-FlpA fusion protein (Trifecta). Chickens were vaccinated by injection at 6 day of age and received a booster injection at 16 days of age. Chickens were challenged with C. jejuni F39011 orally at 20 days of age and euthanised and necropsied at day 27. FIG. 1, from Neal-Mckinney et al, shows the effect of vaccination by injection in this artificial infection trial. Vaccination with FlaA, FlpA and trifecta had the greatest effect on reducing Campylobacter levels resulting in an approximately 3 log reduction in the caeca. Vaccination with Cad F had less of an effect with approx. a 1.5 log reduction in Campylobacter levels, while vaccination with CmeC resulted in only a modest reduction. Thus, Neal-Mckinney et al merely describes the use of certain peptides for vaccination by injection which only results in a 3 log reduction in Campylobacter levels in the caecum. However, vaccination of poultry trough injection in not a commercially viable technique. Neal-Mckinney et al does not demonstrate the efficacy of vaccination using these peptides through the drinking water, which is the common method from distributing vaccines for poultry commercially. It would be expected that vaccination using these peptides through drinking water would be less effective resulting in significantly lower than a 3 log reduction in Campylobacter levels in the caecum. In addition, the peptides used by Neal-Mckinney et al are derived from surface proteins with are extremely immunogenic but the response offered by the immune response is general and un-specific.

Herein, we aim to:
1. To reduce colonization of poultry with Campylobacter jejuni.
2. To reduce the impact that Campylobacter jejuni colonization will have on human gastrointestinal diseases caused by consumption of Campylobacter free chickens.

We will determine if there is a significant decrease in colonization of broilers caecal content and mucosa and to prevent colitis in a rabbit animal model. The measured differences will be in terms of weights, feed conversion, pathogen load in gut mucosa and gut content, integrity of the gastrointestinal mucosa and signs of inflammation at intestinal level. In this way we can determine if this new vaccine formulation could be evaluated further in larger trials.

### SUMMARY OF THE INVENTION

According to the invention, there is provided a polypeptide comprising:
a) an amino acid sequence selected from the group consisting of SEQ ID NOs: 3 and 5, or
b) an amino acid sequence consisting of at least 35 contiguous amino acid residues from SEQ ID NOs: 3 and 5, or
c) an amino acid sequence having a sequence identity of at least 80%, optionally at least 90%, with the amino acid sequence of SEQ ID NOS: 3 and 5, or
d) an amino acid sequence having a sequence identity of at least 80%, optionally at least 90%, with the amino acid sequence consisting of at least 35 contiguous amino acid residues from SEQ ID NOs: 3 and 5,
e) an amino acid sequence having a sequence identity of at least 80%, optionally at least 90%, with an amino acid sequence of at least 10 contiguous amino acid residues, optionally at least 20 contiguous amino acid residues, and comprising amino acid residue 392 from SEQ ID NOs: 3 or 5,
said polypeptide being antigenic in a host.

Optionally, the polypeptide is fused or conjugated to an immunogenic carrier molecule.

Optionally, the polypeptide of the first aspect of the invention i for use as a vaccine.

According to a second aspect of the invention, there is provided antibodies against the polypeptide of the first aspect of the invention for use as a vaccine.

According to a third aspect of the invention, there is provided a vaccine for use in reducing or preventing Campylobacter colonization in a host, the vaccine comprising the polypeptide of the first aspect of the invention and/or antibodies against the polypeptide of the first aspect of the invention. Optionally, the host is poultry, optionally selected from chickens, ducks and turkeys. Further optionally, the vaccine is for use in reducing or preventing Campylobacter jejuni colonization in poultry, optionally selected from chickens, ducks and turkeys.

According to a fourth aspect of the invention, there is provided a vaccine for use in reducing or preventing campylobacteriosis, the vaccine comprising the polypeptide of the first aspect of the invention and/or antibodies against the polypeptide of the first aspect of the invention. Optionally, the vaccine is for use in reducing or preventing campylobacteriosis in humans and domesticated animals such as rabbits, dogs and cats, optionally in humans. Further optionally, the use is prophylactic use or therapeutic use.

The polypeptide of the first aspect of the invention may be expressed from a vector, wherein, optionally, the vector is E. coli or another bacterial organism.

The vaccine may be administered intranasally, intramuscularly, intradermally, subcutaneously, or orally.

The vaccine may be administered in a single dose; or as two or more doses.

The host (optionally poultry, optionally selected from chickens, ducks and turkeys) may receive between about 50-1,000 μg, optionally between about 100-500 μg, further optionally about 240 μg, of the polypeptide of claim 1 or 2, or antibodies against the polypeptide of the first aspect of the invention, per administration.

When the vaccine is administered as two or more doses, the host may receive a priming administration and one or more booster administrations of said polypeptide or of said antibodies against said polypeptide.

There is also provided a vaccine composition comprising the polypeptide of the first aspect of the invention; or antibodies raised against the polypeptide of the first aspect of the invention, in association with a pharmaceutically acceptable vehicle useful for inducing an immune response in a host.

The vaccine composition may be administered intranasally, intramuscularly, intradermally, subcutaneously, or orally. Optionally, the vaccine composition is administered orally, further optionally via drinking water.

The vaccine composition may be administered in a single dose; or as two or more doses.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

### Drawings

FIG. 1A. Coomassie blue stained expressed recombinant antigen has an apparent molecular weight of about 50, 000 Da. However, the recombinant antigen and wild type antigen have the same apparent molecular weight—the Western blot is identical for the wild type antigen (not shown).

FIG. 1B. Computer modelling to illustrate the mutation of Cysteine to Phenylalanine in position 399 by comparing the wild type to the mutated inactivated knockout.

FIG. 1C. Identification of 450 nm absorption in SORET band with CO bound of purified protein. Successive spectra show the transition of the 420 nm peak of the oxidized protein, to a 450 nm peak by exposure of the protein to CO (carbon monoxide) (blue spectra); inactivated protein is green spectrum. The remaining spectra (red and black) are subsequent runs of CO-bound protein over time, to check stability.

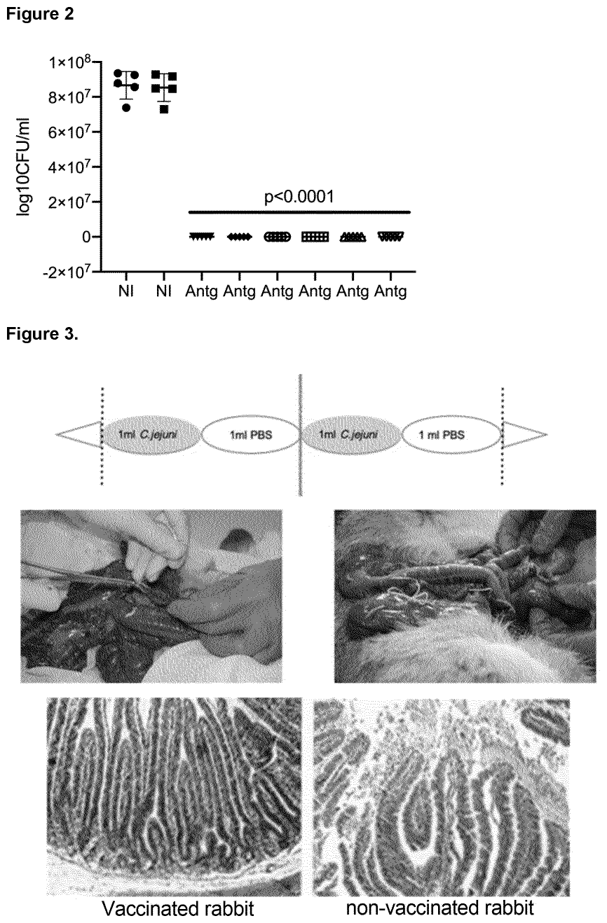

FIG. 2. Levels of Campylobacter colonization in the caecum of artificially infected chicken broilers. Broilers were vaccinated via intramuscular injection with RC039-C399F and then infected with C. jejuni RC039 chicken isolate. NI birds (Control): Broilers did not receive vaccine but were infected with C. jejuni RC039 chicken isolate.

FIG. 3. A rabbit model surgical illustration and histochemistry results showing clear protection at gut level against Campylobacter induced colitis.

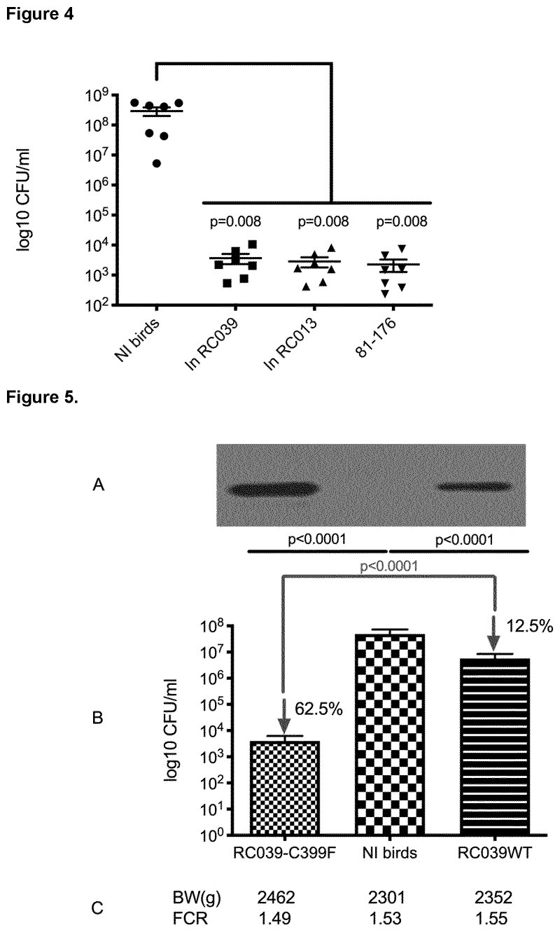

FIG. 4. Levels of Campylobacter colonization in the cecum of artificially infected chicken broilers. NI birds (Control): Broilers did not receive vaccine but were infected with C. jejuni RC039 chicken isolate; In RC039: Broilers were vaccinated via water and then infected with C. jejuni RC039 chicken isolate; In RC013: Broilers were vaccinated via water and then infected with C. coli RC0138; 81-176: Broilers were vaccinated via the drinking water and then infected with C. jejuni 81-176 human isolate.

FIG. 5. Large trial with naturally colonized broilers (1000 broilers in each group). The 3000 broilers were all housed in the same unit. The three groups were separated by plastic transparent sheets. Vaccination was performed with the genetically modified antigen (RC039-C399F) and with the wild type antigen (RC039WT). Panel A shows antigen detection in blood, Panel B quantifies campylobacter (log10 CFU/ml) compared to non-vaccinated broilers (NI), Panel C shows performance indicators—body weight is BW; FCR is feed conversion rate.

### DETAILED DESCRIPTION OF THE INVENTION

As used in the specification and claims, the singular form “a”, “an” and “the” include plural references unless the context clearly dictates otherwise. For example, the term “a polypeptide antigen” includes a plurality of polypeptide antigens, including mixtures thereof.

As used herein, the term “comprising” is intended to mean that the compositions and methods include the recited elements, but do not exclude other elements.

As used herein, the term “polypeptide” is in the present context intended to mean both short peptides of from 2 to 10 amino acid residues, oligopeptides of from 11 to 100 amino acid residues, and polypeptides of more than 100 amino acid residues.

The term “amino acid sequence” is the order in which amino acid residues, connected by peptide bonds, lie in the chain in polypeptides.

The term “adjuvant” has its usual meaning in the art of vaccine technology, i.e. a substance or a composition of matter which is 1) not in itself capable of mounting a specific immune response against the immunogen of the vaccine, but which is 2) nevertheless capable of enhancing the immune response.

Sequence alignment arranges the sequences of DNA, RNA, or protein to identify regions of similarity that may be a consequence of functional, structural, or evolutionary relationships between the sequences. Aligned sequences of nucleotide or amino acid residues are typically represented as rows within a matrix with gaps are inserted between the residues so that identical or similar characters are aligned in successive columns. The term “percent sequence similarity” is the percentage of identical residues (percent identity) between aligned sequences.

An “immunogen” is capable of inducing an adaptive immune response in a host, whose immune system is confronted with the immunogen. As such, immunogens are a subset of the larger genus “antigens”, which are substances that can be recognized specifically by the immune system (e.g. when bound by antibodies or, alternatively, when fragments of the antigens bound to MHC molecules are being recognized by T-cell receptors) but which are not necessarily capable of inducing immunity. However, an antigen is always capable of eliciting immunity, meaning that a host that has an established memory immunity against the antigen will mount a specific immune response against the antigen.

The term “antigen” refers to a compound, composition, or immunogenic substance that can stimulate the production of antibodies or a T-cell response, or both, in an animal, including compositions that are injected or absorbed into an animal. The immune response may be generated to the whole molecule, or to a portion of the molecule (e.g., an epitope or hapten). In the present context, the term “antigen” can include a polypeptide, or a fragment thereof.

An “adaptive immune response” is an immune response in response to confrontation with an antigen or immunogen, where the immune response is specific for antigen determinants of the antigen/immunogen—examples of adaptive immune responses are induction of antigen specific antibody production or antigen specific induction/activation of T helper lymphocytes or cytotoxic lymphocytes.

A “protective, adaptive immune response” is an antigen-specific immune response induced in a subject as a reaction to immunization (artificial or natural) with an antigen, where the immune response is capable of protecting the subject against subsequent challenges with the antigen or a pathology-related agent that includes the antigen. Typically, prophylactic vaccination aims at establishing a protective adaptive immune response against one or several pathogens.

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen.

As used herein, the term “vaccine” includes at least one antigen or immunogen in a pharmaceutically acceptable vehicle useful for inducing an immune response in a host. The immune response in the host can be a cellular and/or antibody-mediated immune response to the vaccine of interest.

The term “immune response” as used herein refers to a response elicited in an animal. An immune response may refer to cellular immunity (CMI), humoral immunity, or may involve both. The present invention also contemplates a response limited to a part of the immune system. Usually, an “immunological response” includes, but is not limited to, one or more of the following effects: the production or activation of antibodies, B cells, helper T cells, suppressor T cells, and/or cytotoxic T cells, directed specifically to an antigen or antigens included in the vaccine of interest. Preferably, the host will display either a therapeutic or protective immunological response, such that resistance to new infection will be enhanced, and/or the clinical severity of the disease reduced. Such protection will be demonstrated by either a reduction or lack of symptoms normally displayed by an infected host, a quicker recovery time, and/or a lowered bacterial titer in the infected host.

Vaccines can be administered in dosages, and by techniques well known to those skilled in the medical or veterinary arts, taking into consideration factors such as the age, sex, pregnancy, weight, species and condition of the recipient animal, and the route of administration. The route of administration can be percutaneous, via mucosal administration (e.g., oral, nasal, anal, vaginal) or via a parenteral route (intradermal, transdermal, intramuscular, subcutaneous, intravenous, or intraperitoneal).

Vaccines can be administered alone, or can be co-administered or sequentially administered with other treatments or therapies. Forms of administration may include suspensions, syrups or elixirs, and preparations for parenteral, subcutaneous, intradermal, intramuscular or intravenous administration (e.g., injectable administration) such as sterile suspensions or emulsions.

Vaccine compositions may be administered as a spray, or mixed in food and/or water, or delivered in admixture with a suitable carrier, diluent, or excipient.

As used herein, the terms “pharmaceutically acceptable carrier” and “pharmaceutically acceptable vehicle” are interchangeable, and refer to a fluid vehicle for containing vaccine antigens that can be injected into a host without adverse effects. Suitable pharmaceutically acceptable carriers known in the art include, but are not limited to, sterile water, saline (including PBS), glucose, dextrose, or buffered solutions. Carriers may include auxiliary agents including, but not limited to, diluents, stabilizers (i.e., sugars and amino acids), preservatives, wetting agents, emulsifying agents, pH buffering agents, viscosity enhancing additives, colouring additives, and the like.

The vaccines can contain auxiliary substances such as wetting or emulsifying agents, pH buffering agents, adjuvants, gelling or viscosity enhancing additives, preservatives, flavouring agents, colours, and the like, depending upon the route of administration and the preparation desired.

Standard pharmaceutical texts, such as “Remington's Pharmaceutical Sciences” (1990), may be consulted to prepare suitable preparations, without undue experimentation.

Vaccine compositions may comprise an adjuvant. An “adjuvant”, as used herein, means one or more substances that enhance the immune response to an antigen(s). The mechanism of how an adjuvant operates is not entirely known. Some adjuvants are believed to enhance the immune response by slowly releasing the antigen, while other adjuvants are strongly immunogenic in their own right, and are believed to function synergistically.

In one embodiment, the vaccine is provided as a single-dose, 1-bottle vaccine. In another embodiment, the vaccine is provided as a multi-dose vaccine. In some embodiments, additional antigens can be added to either the single or the multi-dose vaccine.

The composition may be administered intramuscularly, intradermally, transdermally, subcutaneously, or orally. In another embodiment, the composition is administered in a single dose. In yet another embodiment, the composition is administered as two or more doses.

The vaccine composition may be administered to chickens at 5 days, or older. The vaccine composition may be administered to chickens at least 5 days before their sacrifice.

Vaccine and/or immunogenic compositions according to the present invention can be administered in dosages and by techniques well known to those skilled in the medical or veterinary arts, taking into consideration such factors as the age, sex, weight, species and condition of the recipient animal, and the route of administration. The route of administration can be percutaneous, via mucosal administration (e.g., oral, nasal, anal, vaginal) or via a parenteral route (intradermal, transdermal, intramuscular, subcutaneous, intravenous, or intraperitoneal). Vaccine compositions according to the present invention can be administered alone, or can be co-administered or sequentially administered with other treatments or therapies. Forms of administration may include suspensions, syrups or elixirs, and preparations for parenteral, subcutaneous, intradermal, intramuscular or intravenous administration (e.g., injectable administration), such as sterile suspensions or emulsions. Vaccine compositions according to the present invention may be administered as a spray, or mixed in food and/or water, or delivered in admixture with a suitable carrier, diluent, or excipient such as sterile water, physiological saline, glucose, or the like. The compositions can contain auxiliary substances such as wetting or emulsifying agents, pH buffering agents, adjuvants, gelling or viscosity enhancing additives, preservatives, flavouring agents, colours, and the like, depending upon the route of administration and the preparation desired.

Also contemplated by the present invention are antibodies against the polypeptides of SEQ ID NOS: 1 or 3 (e.g., monoclonal and polyclonal antibodies, single chain antibodies, chimeric antibodies, humanized, human, animal, and CDR-grafted antibodies, including compounds which include CDR sequences which specifically recognize a MOMP of the invention. The term “specific for” indicates that the variable regions of the antibodies of the invention recognize and bind the polypeptide of SEQ ID NO: 3 or 5. Screening assays to determine binding specificity of an antibody of the invention are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6.

Antibodies that recognize and bind fragments of the polypeptides of the invention are also contemplated, provided that the antibodies are specific for a polypeptide of the invention from which the fragment was derived.

As used herein, “antibody” refers to an immunoglobulin molecule that can bind to a specific antigen as the result of an immune response to that antigen. Immunoglobulins are serum proteins composed of “light” and “heavy” polypeptide chains having “constant” and “variable” regions and are divided into classes (e.g., IgA, IgD, IgE, IgG, and IgM) based on the composition of the constant regions. Antibodies can exist in a variety of forms including, for example, as, Fv, Fab′, F(ab′)2, as well as in single chains, and include synthetic polypeptides that contain all or part of one or more antibody single chain polypeptide sequences.

The term “animal” or “host” is, in the present context, in general, intended to denote an animal species (preferably mammalian or avian), such as Homo sapiens, Canis domesticus, Gallus gallus domesticus, Anas platyrhynchos, Cairina moschata, Meleagris gallopavo domesticus, Sus domesticus, Bos taurus, Felis catus etc. and not just one single animal. However, the term also denotes a population of such an animal species, since it is important that the individuals immunized using the vaccine of the invention substantially all will mount an immune response against the polypeptide of the present invention.

Campylobacter is 1 of 4 key global causes of diarrhoeal diseases. It is considered to be the most common bacterial cause of human gastroenteritis in the world. Campylobacter are mainly spiral-shaped, “S”-shaped, or curved, rod-shaped bacteria. Currently, there are 17 species and 6 subspecies assigned to the genus Campylobacter, of which the most frequently reported in human diseases are C. jejuni and C. coli. Other species such as C. lari and C. upsaliensis have also been isolated from patients with diarrhoeal disease, but are reported less frequently. Campylobacteriosis is the disease caused by the infection with Campylobacter.

Campylobacter species are widely distributed in most warm-blooded animals. They are prevalent in food animals such as poultry, cattle, pigs, sheep and ostriches; and in pets, including cats and dogs. The bacteria have also been found in shellfish.

The main route of transmission is generally believed to be foodborne, via undercooked meat and meat products, as well as raw or contaminated milk. Contaminated water or ice is also a source of infection. A proportion of cases occur following contact with contaminated water during recreational activities.

Campylobacteriosis is a zoonosis, a disease transmitted to humans from animals or animal products. Most often, carcasses or meat are contaminated by Campylobacter from faeces during slaughtering. In animals, Campylobacter seldom causes disease.

The relative contribution of each of the above sources to the overall burden of disease is unclear but consumption of undercooked contaminated poultry is believed to be a major contributor. Since common-source outbreaks account for a rather small proportion of cases, the vast majority of reports refer to sporadic cases, with no easily discernible pattern.

As used herein, the term Campylobacter colonization has been used in relation to those hosts not having a disease phenotype (or showing symptoms). The main focus is in relation to “food animals”—animals whose meat or milk is intended to be consumed by those susceptible to campylobacteriosis.

The invention is intended to reduce or prevent Campylobacter colonization in food animal hosts, such as poultry. Reducing or preventing colonization is important in poultry, such as chickens, ducks and turkeys. This will reduce the incidence of Campylobacter in the meat, thereby reducing zoonotic infection (infection between species—from humans to animals) of those who ingest the meat.

The invention is also intended to reduce or prevent campylobacteriosis in a host having a disease phenotype, such as humans and domesticated animals. Campylobacteriosis is an infection caused by bacteria acquired from contaminated food (such as poultry that isn't fully cooked) and water. It causes diarrhoea. It is also called campylobacter, campylobacter infection, or campylobacteriosis gastroenteritis. Infants and children have a greater chance than adults for campylobacter infection, but it can strike anyone at any age. Men are also more likely than women to get infected. It's more common in summer than winter.

Domesticated animals are animals that have been selectively bred and genetically adapted over generations to live alongside humans. Animal domestication includes domestication for companionship (rabbits, dogs and cats).

Such vaccines according to the invention, for use to reduce or prevent campylobacteriosis, may either be prophylactic (i.e. to prevent infection) or therapeutic (i.e., to treat disease after infection).

A vaccine can, in a very simple form, comprise just isolated polypeptide against Campylobacter and possibly a diluent. Such a diluent can be added to dilute the antiserum if the amount of antibody titre is too high. The diluent can be as simple as distilled water, or physiological salt solution. Actually any pharmaceutically acceptable diluent can be used.

### Material and Methods

### Antigen Construction and Expression

Campylobacter jejuni 81-176 wild type was grown on Mueller-Hinton (MH) Agar or in MH Broth (OXOID, UK) at 37° C. for 48 h under microaerobic conditions (5% CO₂, 5% O₂, 90% N₂).

The E. coli strains Nova Blue (endA1 hsdR17 (r_{K12}⁻m_{K12}⁺) supE44 thi-1 recA1 gyrA96 relA1 lac F′[proA⁺B⁺lacl^{q}ZΔM15::Tn10] (Tet^{R})) or TUNER (F⁻ ompT hsdS_{B} (r_{B}⁻m_{B}⁻) gal dom lacY1(DE3) pLacl (Cam^{R})) were maintained in either LB Broth or agar with appropriate antibiotics.

For antibody production, the Cj1411c gene was amplified from C. jejuni 81-176 genomic DNA using primers P450 FOR (ATGAGTGAATGCCCCTTTTTTCCAAAACCTTATAAAAATAAAGC (Sequence given 5′ to 3′; SEQ ID NO: 1) and P450 REV (TCAGTGGTGGTGGTGGTGGTGTAGCTTTCTTTTGCTAAATTTTAT) (Sequence given 5′ to 3′; SEQ ID NO: 2). Following amplification by PCR, the recombinant DNA fragment was ligated into the EcoRV site of the pETBlue1 vector. For initial cloning, the ligated vector was transformed in E. coli Nova Blue and for overexpression in E. coli TUNER. The overexpression strain was grown in Terrific Broth with carbenicillin/chloramphenicol at 37° C. until OD₆₀₀=0.5 and induced with 1 mM IPTG overnight at room temperature. Bacterial pellets were harvested and lysed in 25 mM Tris pH 7.5, 250 mM NaCl, 25 mM Imidazole, 1 mM DTT, 0.1% Triton X100 and protease inhibitors. E. coli lysates were loaded onto a HisTrap™FF column and eluted with 25 mM Tris 7.5, 250 mM NaCl, 0.1% Triton, 1 mM DTT on an AKTApurifier®. Collected samples were desalted and concentrated on Centricon® devices with a MW 10.000 cut-off and stored in 25 mM Tris-HCL pH 7.5 at −80° C.

Protein identity, purity and phosphorylation state was analyzed by Coomassie blue (see FIG. 1 for the recombinant peptide of SEQ ID NO: 3). The protein was eluted with a gradient of 25 mM Tris pH 7.5, 250 mM NaCl, 250 mM Imidazole, 1 mM DTT, 0.1% Triton X100 and concentrated to 5-10 ml. In order to obtain a higher purity, the concentrated sample was additionally purified on a Superdex S200 FPLC column in 25 mM Tris pH 7.5, 250 mM NaCl, 1 mM DTT, 0.1% Triton X100. The eluted protein was concentrated to a volume of approximately 5 ml (100 μM).

Substitution of Cysteine at position 399 with Phenylalanine (F), within the active site, was carried out by Capra Science (Sweden), to yield the recombinant peptide of SEQ ID NO: 3.

Both purified proteins were used for antibody production at Capra Science (Sweden).

Cytochrome P450 from Campylbacter jejuni is a polypeptide of 453 amino acids:

RefSeq Selected Product: WP_002780438.1, 453 amino acids

Taxonomic Group: e-proteobacteria

### Antigen Sequence—Cytochrome P450—Wild Type

(SEQ ID NO: 5)
MSECPFFPKPYKNKASTLLTFLLKRRSWLDGLYERSYKMQTGYVKMPNF
DLYVINDTKEVKRMMVDEVREFPKSAFLHELLSPLLGESIFTTNGEVWK
KQRELLRPSFEMTRINKVFNLMSEAVADMMDRFSKYPNHAVIEVDEAMT
FITADVIFRTIMSSKLDEEKGKKILNAFVTFQEQSVHTAMRRMFRFPKW
LSYVLGDCKRAKAGDVIRQVLSDIIKPRYDMADNAEFEDILGSLLLVVD
ADTNKRFSFEEILDQVAMLFLAGHETTASSLTWTLYLLSLYPKEQEKAY
EEITQVLQGGVIEISHLRQFKYLTNIFKESLRLYPPVGFFAREAKKDTQ
VRDKLIKKGSGVVIAPWLIHRHEEFWTNPHGFNPSRFEGEYKKDAYLPF
GVGERICIGQGFAMQEAILILANILKTYKLELEEGFVPDVVGRLTVRSA
NGMRIKFSKRKL

The active site, within SEQ ID NO: 5 (starting at position 392), is thought to be FGVGERICIG (SEQ ID NO: 6).

### Antigen Sequence—Cytochrome P450—Cysteine at Position 399 was Substituted with Phenylalanine (F)

(SEQ ID NO: 3)
MSECPFFPKPYKNKASTLLTFLLKRRSWLDGLYERSYKMQTGYVKMPNF
DLYVINDTKEVKRMMVDEVREFPKSAFLHELLSPLLGESIFTTNGEVWK
KQRELLRPSFEMTRINKVFNLMSEAVADMMDRFSKYPNHAVIEVDEAMT
FITADVIFRTIMSSKLDEEKGKKILNAFVTFQEQSVHTAMRRMFRFPKW
LSYVLGDCKRAKAGDVIRQVLSDIIKPRYDMADNAEFEDILGSLLLVVD
ADTNKRFSFEEILDQVAMLFLAGHETTASSLTWTLYLLSLYPKEQEKAY
EEITQVLQGGVIEISHLRQFKYLTNIFKESLRLYPPVGFFAREAKKDTQ
VRDKLIKKGSGVVIAPWLIHRHEEFWTNPHGFNPSRFEGEYKKDAYLPF
GVGERIFIGQGFAMQEAILILANILKTYKLELEEGFVPDVVGRLTVRSA
NGMRIKFSKRKL

The site corresponding to the wild type active site, within SEQ ID NO: 3 (starting at position 392), is thought to be FGVGERIFIG (SEQ ID NO: 4).

### Western Blotting

Proteins were separated on 10% SDS polyacrylamide gels and transferred to 0.45 μm nitrocellulose membrane (Millipore). Following WB (Western blot), the membranes were blocked in 3% BSA+0.05% Tween for 30 minutes. Primary antibodies against Cj1411c P450 (rabbit polyclonal), anti-His (sc-53073, Santa Cruz Biotechnology) were used. CadF and Fur (rabbit polyclonal antibodies) have been used to validate the purity of our membrane fractionations. Following washing in PBS-TWEEN, the membranes were incubated with the secondary antibodies (goat anti-rabbit—#7074S, Cell Signaling; goat anti-mouse—sc-2031, Santa Cruz). Detection was performed with the PIERCE chemiluminiscence kit (Thermo, Fisher Scientific). Densitometry was performed using Fiji Image J software.

Objectives

To obtain data comparing the following in vaccinated versus non-vaccinated groups:
1. Campylobacter jejuni quantification in broiler caecum.
   Method: The entire caecum (containing both adherent and luminal bacteria) is harvested from broilers for the purpose of microbiota analysis. The broiler caeca are dissected aseptically and immediately snap frozen (liquid nitrogen) until processing. Caecal DNA is extracted using the QIAamp DNA Stool Mini Kit according to the manufacturer's instructions (Catalog #51604). Genomic DNA concentration is determined at a wavelength of 260 nm using a NanoDrop. For qPCR analysis, DNA samples are diluted in sterile water to a concentration of 1 ng/μl. The real time PCR is performed on a part of the prepared volume.
2. Levels of antibody production in the serum of broilers and rabbits
   Method: The antibody levels in blood serum are measured using ELISA method as previously described (Neal-Mckinney, et al., 2014).
3. Histological investigation of luminal mucosa for signs of colitis in Rabbits—see FIG. 3
   Method: Following artificial infection, the rabbit intestinal tissue is subjected to histology for localized inflammation as described below.
4. Levels of antibody in tissues
   Method: The antibody levels are measured using ELISA method as previously described (Neal-Mckinney, et al., 2014).
5. Quantification of feed conversion rates in vaccinated versus non-vaccinated in broilers—see FIG. 5
   Method: records of feed amounts are kept daily and the feed conversion rates are calculated at the end of the experiment.

### Broilers and Rabbit Accommodation

Chicken broilers are housed in special isolators for experimental challenge. Rabbits are housed in a specialised rabbit cages or floor pens.

### Vaccination via Injection or Water Protocol

The polypeptides were HIS-tagged to facilitate purification; it is not thought that HIS-tagging plays a role in vaccine efficacy. At 6 days of age, the chicks are injected with 240 μg of the HIS-tagged P450 full-length polypeptides (either wild type or recombinant). At 16 days of age, a booster injection is given with 240 μg of full-length P450 polypeptides (either wild type or recombinant). All polypeptides (antigens), for injection, are emulsified in Montanide ISA 70 VG (Seppic, Paris, France), a commonly used adjuvant for poultry vaccines, at a ratio of 30% antigen and 70% adjuvant. Montanide ISA 70 VG is a mineral oil-based solution incorporating a highly refined mannitol/oleic acid emulsifier. Seppic recommend that the peptide is emulsified with ISA 70 at a 30:70 ratio (w: w; polypeptide: adjuvant) as recommended by the manufacturer. For the primary and booster injections, 200 mL of polypeptide/adjuvant mixture are injected into the left and right breast muscle.

For water vaccination, the polypeptide is diluted in PBS, at a ratio of 30% (w) antigen and 70% (v) PBS (Phosphate Buffer Saline) and added to the drinking water on day 6 for up to 2 hours (i.e. 30 g antigen is diluted to 100 ml in PBS; or 30% (w/v)). The water vaccination follows the protocol below:
1. 8 am to 8.45 am—Feed and water.
2. 8.45 am—Turn water off and let birds drink remaining water in drinkers (20 minutes approximately).
3. 9.05 am—Raise drinkers or lines out of the reach of the birds.
4. 9.05 am to 10.05 am—Water withdrawal time. Make up vaccine to dose the volume of water that will be drunk in about 1½-2 hours, at a dosage of 240 μg of antigen per broiler
5. 10.10 am—Drain residual water from each line/bell (1 liter/3 meters of line) until milky/white colored water solution appears and then lower lines/bells to the birds.
6. 10.10 am to 12.10 pm—Vaccination period. Walk flock twice during vaccination, check flow of vaccine along all water lines and to all drinkers.
7. 12.10 pm—Turn on mains water after vaccination. Clean water proportioner.

### Campylobacter Jejuni Artificial Challenge Experiment

Chicks are challenged with 26×10⁸ colony-forming units (CFU) of C. jejuni orally at 20 days of age. The birds are humanely euthanized by carbon dioxide asphyxiation and necropsied at day 27. One caecum from each bird is weighed and stomached (using a stomacher mixer) in an equal volume of MH broth (1 mL broth per gram of caecal contents). The caecal contents were then serially diluted and plated on Campy-Cefex Agar for enumeration. Blood is aspirated directly from the heart of necropsied chickens to obtain serum for determining presence/absence of antibody levels.

### Ligated Rabbit Ileal Loops

A disease phenotype model was investigated using a ligated rabbit ileal loop model as described by Newell et al, 2001. The protocol may be modified depending on particular circumstances and on the expertise of the veterinary surgeon.

Two rabbits were used in this study. One rabbit was vaccinated by injection. 72 hours later, the two rabbits were sedated in familiar surroundings (rabbit cage) to minimise stress. Rabbits were sedated by intramuscular injection of acepromazine and butorphanol (1 mg/kg+1 mg/kg). When sedated, rabbits were transported to the operating theatre and fully anesthetised by intravenous injection of fentanyl/fluanisone (Hypnorm)+diazepam (0.3 ml/kg im+1-2 mg/kg iv, im or ip). The anaesthetic and analgesic agents used were as agreed with Consultant Veterinarian and Named Veterinary surgeon. Under full anaesthesia, four ileal loops (5 cm in length) were isolated and ligated in each of the two rabbits. Two loops in each rabbit were injected with PBS. The other two loops in each rabbit were injected with C. jejuni RC039 using 1 ml of culture at OD₆₀₀=0.3 in PBS (pH 7.4). After closure of the abdomen, rabbits were placed in cages for 6 hours. If necessary, a suitable opioid analgesic and non-steroidal anti-inflammatory drug may be administered. Analgesic treatment may include opioid drugs such as buprenorphine (0.01-0.05 mg/kg, SC, IM, or IV, bid-tid) or butorphanol (0.05-0.4 mg/kg, SC or IM, bid-tid), or NSAIDs such as carprofen (1.5 mg/kg, PO or SC, bid), flunixin (0.5-2 mg/kg/day, PO, deep IM, or IV, for no more than 3 days), or meloxicam (1 mg/kg/day, PO or SC) before any surgical intervention, as agreed with Veterinary Consultant. Rabbits were sacrificed by intravenous injection of sodium pentobarbital (120 mg/kg). Fluid accumulated in each loop was collected separately and processed further for bacteria quantification and the intestinal tissue for histological determination of colitis and inflammation. The surgical procedure is performed by an experienced Veterinary Surgeon.

### Feeding of Birds

Once the birds are in the isolators, they are monitored by an Animal Service staff at least twice a day, food is provided in poultry feeders and fresh water supply is constant (automated system).

### Laboratory Analysis

The laboratory analyses chosen in this study are widely used and are recognised as suitable for the evaluation of the effective parameters of vaccine efficacy. These analyses measure the efficacy of the vaccine primarily by determining antibody responses or viral loads as strong antibody responses or reduced viral loads are recognised as indicative of successful vaccination.

To analyse the rabbit tissue, the samples are processed embedded in paraffin and cut into 5 μm sections by microtome. Sections are deparaffinized with xylene (10 min) and hydrated using an ethanol gradient (100%—6 min, 95%—6 min, 70%—3 min) to distilled water (3 min). Samples are then stained with hematoxylin (7 min) and eosin (3 min). The stained slides are dehydrated (70% IMS—1 min, 95% IMS—2 min, 100% IMS—2 min), cleared in xylene and mounted in DPX. Slides are analyzed using a Leica DFC300x camera and the IM50 imaging software.

### Results

### Purification of the Recombinant Antigen

This experimental design aims to test if a polypeptide of the present invention, such as SEQ ID NO: 3 or 5, would generate enhanced protection (i.e., a reduction in C. jejuni colonization). As described in Material and Methods, we have mutated the cysteine 399 (C) to a phenylalanine (F), to yield SEQ ID NO: 3. Following expression in E. coli, as described in Material and Methods, FIG. 1 shows the successful purification of the purified recombinant polypeptide of SEQ ID NO: 3 following Coomassie blue staining of SDS gels of the recombinant RC039-C399F. The purification method is also described in detail in Material and Methods. The successful antigen expression and purification is shown in FIG. 1 panel A.

Very importantly, the substitution of C to F has not affected structurally the protein as indicated using computer modelling in FIG. 1 panel B. The inactivation was also tested via transition to oxidation—see FIG. 1 panel C.

FIG. 1. Panel A shows that the expressed recombinant polypeptide of SEQ ID NO: 3 has an apparent molecular weight of about 50,000 Da. Panel B shows computer modelling to illustrate the mutation of Cysteine to Phenylalanine in position 399 by comparing the wild type to the mutated inactivated knockout. Panel C confirms the protein is a P450 cytochrome (only such proteins have this spectral shift from 420 nm to 450 nm when exposed and bound to carbon monoxide (CO)). The blue spectra monitor the shift from 420 nm to 450 nm after exposure to CO. Once the spectrum has reached 450 nm, we continued to run spectra over time to monitor stability (red and black spectra). The green spectrum was heat treated inactivated protein. Panel C shows identification of 450 nm absorption in SORET band with CO bound of purified protein. Successive spectra show the transition from the 420 nm peak, of the oxidized protein, to 450 nm peak by exposure of the protein to the CO (blue spectra); inactivated protein is green spectrum).

### In Vivo Testing of Efficacy in Preventing Colonisation following Artificial Infection with C. jejuni RC039 and Intra-Muscular Administration of the antigen

In order to test the efficacy of the polypeptide of SEQ ID NO: 3 or 5 in preventing colonisation, we tested, as described in Material and Methods, 8 broilers artificially infected with C. jejuni RC039. 2 non vaccinated broilers (NI) were used as controls, while the other 6 broilers were vaccinated via intra-muscular administration of the polypeptide of SEQ ID NO: 5—antigen (Antg). All 8 broilers were kept in the same enclosure, but separated into different pens. As shown in FIG. 2, all 6 broilers exposed to vaccination were not colonised with C. jejuni RC039 (p<0.0001).

### The Impact of Vaccination on the Epithelial Gut Structures as a Consequence of Campylobacter Infection

In the absence of a disease phenotype in chicken broilers and in order to prove the effect on the gut epithelium of colonisation, we have used the rabbit ileal loop infection model as described in the Materials and Methods. As shown in FIG. 3, the epithelial crypts of the ileal loops infected with C. jejuni in the non-vaccinated rabbit were clearly damaged by infection. In comparison, the epithelial crypts of ileal loops in the vaccinated rabbit that was subsequently infected with C. jejuni showed no crypt damage. These results clearly show that vaccination with the polypeptide of SEQ ID NO: 3 provides protection against Campylobacter infection in a rabbit ileal model.

FIG. 3 shows a rabbit model surgical illustration and histochemistry results showing clear protection at gut level against Campylobacter induced colitis.

### In Vivo Testing of Efficacy in Preventing Colonisation following Artificial Infection with C. jejuni RC039 and Administration of the Antigen via Drinking Water

In order to quantify the efficacy of the polypeptide vaccine on different Campylobacter species, we tested the effect of vaccination via drinking water in preventing the colonisation of artificially infected broilers. For this study, seven broilers were artificially infected in each of the 4 groups. A control group was infected with Campylobacter jejuni (RC039) but not vaccinated (NI birds). The other three groups were infected with Campylobacter jejuni (RC039) chicken isolate, Campylobacter coli (RC013) chicken isolate, and Campylobacter jejuni 81-176 human isolate respectively. All three of these groups received vaccination with the polypeptide of SEQ ID NO: 3, via the drinking water, before being challenged with the identified Campylobacter species.

FIG. 4 shows levels of Campylobacter colonization in the caecum of artificially infected chicken broilers. NI birds (Control): Broilers were infected with C. jejuni RC039 chicken isolate and did not receive vaccine; In RC039: Broilers were vaccinated via water and then infected with C. jejuni RC039 chicken isolate; In RC013: Broilers were vaccinated via water and then infected with C. coli RC0138; 81-176: Broilers were vaccinated via the drinking water and then infected with C. jejuni 81-176 human isolate.

The results presented in FIG. 4 clearly show that vaccination via drinking water significantly protects against all three strains used (p=0.008).

### Efficacy of Water Vaccination in Naturally Colonised Broilers in a Farm Set Up Using 1,000 Broilers in Each of 3 Groups

Next, we have investigated the effect of water mediated vaccination against natural colonisation of broilers with Campylobacter jejuni using each of the polypeptides of SEQ ID NOS: 3 and 5.

Three thousand male broiler chickens (Ross 308) were housed in the experimental farm on wood shaving bedding. 1,000 birds were used as a non-vaccinated control (NI birds), 1,000 birds were vaccinated with the wild type polypeptide of SEQ ID NO: 5 (RC039WT) and 1,000 birds were vaccinated with the recombinant polypeptide of SEQ ID NO: 3 (RC039-C399F). The three groups were divided using plastic transparent sheets. The temperature in the isolation unit was kept between 22-25° C. and thermostatically controlled. Broilers were fed ad libitum with standard diets (starter/grower/finisher). Broilers were euthanized at day 35 and C. jejuni was enumerated by analysing the cecum content for plate counting.

FIG. 5 shows the levels of Campylobacter colonization in the caecum of naturally colonised chicken broilers vaccinated (with either the recombinant polypeptide of SEQ ID NO: 3 (RC039-C399F) or the wild type polypeptide of SEQ ID NO: 5 (RC039WT) via water (FIG. 5). The levels of antigen production in blood is indicated by Western blotting (panel A). Our results also show that the levels of Campylobacter colonization in the caecum of naturally colonised chicken broilers were significantly lower (p<0.0001) when vaccination was done with the polypeptide of either of SEQ ID NO: 3 or 5. Specifically, a 2 Log CFU/ml decrease was noted when the polypeptide of SEQ ID NO: 5 was used compared to approximately 4 Log CFU decrease when the recombinant polypeptide of SEQ ID NO: 3 was used. Moreover, we show, in FIG. 5 panel C, that the performance parameters—body weight (BW) and feed conversion rate (FCR) were improved when the presence of Campylobacter was reduced.

We have described the use of the polypeptide of SEQ ID NO: 3 or 5 as a vaccine against Campylobacter.

We have demonstrated the efficacy of this vaccine through:
1. An artificial infection trial with vaccination by injection.
   In an artificial infection trial, we demonstrated that vaccination with RC039-C399F via injection could reduce Campylobacter level in the caeca below detectable levels (FIG. 2).
2. An artificial infection trial with vaccination through the drinking water.
   In an artificial infection trial, we demonstrated that using vaccination with RC039-C399F through the drinking water, we achieved a 5 log reduction in Campylobacter levels in the caecum when tested against 2 separate strains of C. jejuni and a strain of C. coli.
3. A natural infection trial with vaccination through drinking water.
   In a natural infection trial with 1000 broilers in each group vaccinated with RC-39-C399F through the drinking water, we demonstrated a 4 log reduction in Campylobacter levels in the caecum of the vaccinated birds versus the unvaccinated control birds (FIG. 4).

We have compared the vaccine of the present invention with that disclosed in Neal-Mckinney et al (2014):

| Neal- Mckinney et al peptides | The present invention |
|---|---|
| Derived from surface proteins | Derived from a protein involved in |
| | bacterial respiration |
| Vaccination via injection | Vaccination via injection reduced |
| demonstrated 3 log reduction | Campylobacter colonisation below |
| | detectable levels (LOD of 10³/g) |
| Tested against one strain of | Tested against multiple strains of |
| Campylobacter jejuni | C. jejuni and one strain of |
| | Campylobacter coli |
| Not tested in a natural infection | Tested in natural infection trial with |
| trial | 1000 broilers in each group. |
| Not tested via drinking water | Tested with vaccination via drinking |
| | water in artificial infection trial |
| | (5 log reduction in Campylobacter |
| | colonisation) and in natural infection |
| | trial (4 log reduction in |
| | Campylobacter colonisation) |
| Likely not commercially viable as | Commercially viable |
| unlikely to achieve at least 3 log | |
| reduction in Campylobacter | |
| colonisation at commercial scale | |
| when applied through drinking | |
| water | |

The amount of challenge polypeptide and of booster polypeptide, as used in Neal-Mckinney et al (2014), was identical to that used herein −240 μg. The timing of the vaccination protocol was also identical. However, Neal-Mckinney et al (2014) solely vaccinated by injection whereas, herein, we have demonstrated efficacy both by injection and via drinking water.

FIG. 4 illustrates a large trial with naturally colonized broilers (1,000 broilers in each group). FIG. 4, panel B, quantifies Campylobacter (log10 CFU/ml) compared to non-vaccinated broilers (NI) and demonstrates, with a statistical significance of less than 0.0001, reduced Campylobacter colonisation (measured by CFU—limit of detection (LOD) of 10³/g) for a polypeptide of SEQ ID NO: 3 or 5.

### Discussion

To date, only a limited number of Campylobacter vaccine strategies have been examined in poultry. A goal of poultry industry is to reduce the incidence of human infection and ensure the supply of safe food to consumers. The presence of C. jejuni in the caeca of poultry at slaughter results in cross-contamination during processing. Therefore, it should be possible to reduce the incidence of human infection by lowering the number of C. jejuni in birds bound for the food supply. The vaccination strategy described herein resulted in a greater than 2 log10 reduction in the level of C. jejuni colonization. According to risk analysis studies, this reduction in colonization should significantly lower the public health risk.

One advantage of this study is that the vaccine was tested on birds challenged with a highly virulent, chicken isolate (RC039) of C. jejuni and C. coli (RC013), thus the vaccine may be effective against C. jejuni strains with varying peptide sequences. Moreover, the effectiveness of this vaccination strategy was also strengthened by the tests also done on naturally colonised birds, in farm setup. Our work clearly demonstrated the efficiency of the vaccine in an industrial environment.

An important consideration in the vaccination strategy utilized in this study is that the polypeptide of the present invention was delivered both via intramuscular injection and via drinking water in order to test the most efficient way of administering the vaccine. This has resulted in a robust investigation with positive results in lowering Campylobacter levels in farmed broilers and also ensuring efficient vaccination in large scale flocks. Water vaccination was proven to be an efficient alternative peptide delivery method which may also make the vaccination of large numbers of chickens more feasible, as the intramuscular vaccination of thousands of birds would be very labour intensive and costly.

Moreover, we have shown that vaccination with a polypeptide of SEQ ID NO: 3 leads to protection also in animal infection model (rabbit ileal loop) resulting in the production of a protective mucosal response as evidenced by a reduction in C. jejuni colonization. The microscopical analysis of ileal tissue of infected and vaccinated rabbits clearly shows no signs of villus alteration compared to the non-infected rabbits where signs of intestinal colitis were observed. We have demonstrated a reduced incidence of infection in animals with a disease phenotype—in the case, a rabbit model.

We have demonstrated a significant effect against naturally and artificially occurring Campylobacter infections in poultry using a vaccine comprising a polypeptide of the present invention, such as SEQ ID NO: 3 or 5. Vaccine-mediated protection caused 62% reduction in colonisation in our animal trials. Our results demonstrate that a significant vaccine-mediated reduction in Campylobacter-colonisation in poultry has been achieved herein, using a polypeptide of the present invention.

This vaccine of the present invention uses proteins involved in bacterial respiration to create an immune response and ultimately target campylobacter colonisation in broiler chickens. This vaccine is based on a protein involved in vital metabolic processes which, once attacked by antibodies produced by the immune system, will result in bacterial death or in reduced pathogen abilities to maintain and colonise the avian gut. This has allowed the production of specific vaccine targets without contamination by similar products that might be synthesized in the original organism. Another advantage of this technology is that only biologically active proteins are produced allowing a tighter control of the effective dosage required.

We have demonstrated a significant effect against naturally and artificially caused Campylobacter colonization in poultry using a vaccine of the present invention. Vaccine-mediated protection caused 62% reduction in colonization in our animal trials. Our results demonstrate that a significant vaccine-mediated reduction in Campylobacter-colonization has been achieved. This will reduce incidence of Campylobacter on the meat, thereby reducing zoonotic infection passed to those who eat or handle the infected meat.

### REFERENCES

Kelly, D. J. (2001). The physiology and metabolism of Campylobacter jejuni and Helicobacter pylori. Symp Ser Soc Appl Microbiol(30), 16S-24S.
Neal-Mckinney, J. M., Samuelson, D. R., Eucker, T. P., Nissen, M. S., Crespo, R., & Konkel, M. E. (2014). Reducing Campylobacter jejuni colonization of poultry via vaccination. PLoS One, 9(12), e114254.
Newell, D. G. (2001). Animal models of Campylobacter jejuni colonization and disease and the lessons to be learned from similar Helicobacter pylori models. Symp Ser Soc Appl Microbiol(30), 57S-67S.
Sima, F., Stratakos, A. C., Ward, P., Linton, M., Kelly, C., Pinkerton, L., Stef, L., Gundogdu, O., Lazar, V., & Corcionivoschi, N. (2018). A Novel Natural Antimicrobial Can Reduce the in vitro and in vivo Pathogenicity of T6SS Positive Campylobacter jejuni and Campylobacter coli Chicken Isolates. Front Microbiol, 9, 2139.
Song, Y., Malmuthuge, N., Steele, M. A., & Guan, L. L. (2018). Shift of hindgut microbiota and microbial short chain fatty acids profiles in dairy calves from birth to pre-weaning. FEMS Microbiol Ecol, 94(3).

1. A polypeptide comprising the amino acid sequence of SEQ ID NO:4 said polypeptide being antigenic in a host.

2. The polypeptide of claim 1 wherein the polypeptide is fused or conjugated to an immunogenic carrier molecule.

3-4. (canceled)

5. A vaccine formulation for use in reducing or preventing Campylobacter colonization in a host, the vaccine comprising a polypeptide comprising:
a) an amino acid sequence selected from the group consisting of SEQ ID NOs: 3 and 5, or
b) an amino acid sequence consisting of at least 35 contiguous amino acid residues from SEQ ID NOs: 3 and 5, or
c) an amino acid sequence having a sequence identity at least 90%, with the amino acid sequence of SEQ ID NOs: 3 and 5, or
d) an amino acid sequence having a sequence identity of at least 80%, optionally at least 90%, with the amino acid sequence consisting of at least 35 contiguous amino acid residues from SEQ ID NOs: 3 and 5,
e) an amino acid sequence having a sequence identity of at least 80%, optionally at least 90%, with an amino acid sequence of at least 10 contiguous amino acid residues, optionally at least 20 contiguous amino acid residues, and comprising amino acid residue 392 from SEQ ID NOs: 3 or 5,
said polypeptide being antigenic in a host, and wherein said vaccine formulation optionally comprises an adjuvant.

6. A method comprising administering the vaccine formulation of claim 5 to a host to stimulate an immune response against Campylobacter bacteria, wherein the host is a human or a rabbit, dog or cat or poultry, the poultry optionally selected from chickens, ducks and turkeys.

7. The method of claim 6 wherein the host is the poultry, and wherein administering the vaccine formulation to the host reduces or prevents Campylobacter jejuni colonization in the poultry.

8. The method of claim 6, wherein administering the vaccine formulation inhibits or prevents development of campylobacteriosis within the host.

9-11. (canceled)

12. The method of claim 6, wherein the vaccine formulation is administered intranasally, intramuscularly, intradermally, subcutaneously, or orally and wherein oral administration is optionally via drinking water

13. (canceled)

14. The method of claim 6, wherein the vaccine formulation is administered in a single dose; or as two or more doses.

15. The method of claim 6, wherein the host receives 50-1,000 μg of the polypeptide per administration.

16. The method of claim 6, wherein the vaccine formulation is administered as two or more doses, wherein the host receives a priming administration and one or more booster administrations of said polypeptide.

17. The vaccine formulation of claim 5, further comprising pharmaceutically acceptable vehicle useful for inducing an immune response in a host.

18-22. (canceled)

23. The vaccine formulation of claim 5, wherein the vaccine formulation comprises the adjuvant.

24. The vaccine formulation of claim 5, wherein the polypeptide comprises the sequence of SEQ ID NO:4.

25. The method of claim 6, wherein the polypeptide comprises the sequence of SEQ ID NO: 4.

26. Isolated antibodies raised against a polypeptide comprising the sequence of SEQ ID NO: 4.
